(19) United States
(12) Patent Application Publication
(10) Pub. No.: US 2022/0331471 A1
(43) Pub. Date: Oct. 20, 2022

(54) SYSTEMS AND METHODS FOR DETECTING AND CAPTURING VIRUSES AND DISINFECTING AIR CONTAINING VIRUSES

(71) Applicant: Andrew T. Wang, Henderson, NV (US)

(72) Inventor: Andrew T. Wang, Henderson, NV (US)

(21) Appl. No.: 17/456,872

(22) Filed: Nov. 29, 2021

Related U.S. Application Data
(60) Provisional application No. 63/118,838, filed on Nov. 27, 2020.

(51) Int. Cl. A61L 9/14; A61L 9/22

(52) U.S. Cl. CPC A61L 9/145; A61L 9/22; A61L 2209/13; A61L 2209/213

(57) ABSTRACT

Systems and methods for detecting, capturing and/or disinfecting viruses. A water trap system includes a water-filled container through which air is introduced into a water-filled container. Charged viruses, bacteria and other contaminants become trapped in the water-filled container as they are entrapped by oppositely charged atoms (e.g., oxygen). Another device incorporates an electric field created by a pair of electrodes with an outer surface heated to a threshold temperature significant enough to disinfect viruses that come into contact therewith. Another device is a portable inhaler for detecting the polarity of airborne viruses and counting the positive and negative electrical charges of airborne viruses with tolerances of a few charged particles per cubic centimeter by sampling several hundred cubic centimeters of air per second. Consequently, the portable inhaler is capable of quickly and efficiently detecting whether a person has contracted a virus (e.g., COVID-19 virus).

### CROSS-REFERENCE

This application claims priority to U.S. Patent Application No. 63/118,838 filed Nov. 27, 2020 and which is incorporated by reference herein for all purposes.

### FIELD OF THE INVENTION

The embodiments of the present invention relate to viruses generally and how to detect their presence, capture them and/or kill them (i.e., disinfect the air carrying them).

### BACKGROUND

Many destructive outbreaks in human history, such as the flus of 1918, SARS, MERS, Ebola, and COVID-19 are all caused by viruses.

Table 1 below summarizes the number of hours different coronaviruses survive in air and on different surfaces.

TABLE 1
# Hours Coronaviruses Survive in Air on Different Surfaces

| | SARS- | SARS- | MERS- | |
|---|---|---|---|---|
| | CoV-2 | CoV-1 | CoV-1 | HCoV |
| AIR | 3 | 3 | — | — |
| PAPER | — | 96 | — | — |
| CARDBOARD | 24 | 8 | — | — |
| WOOD | — | 96 | — | — |
| COPPER | 4 | 8 | — | — |
| GLASS | — | 96 | — | 120 |
| CERAMIC | — | — | — | 120 |
| PLASTIC | >72 | 216 | 48 | 144 |
| STEEL | 48 | 48 | 48 | 120 |

Covid-19 is a relatively new species to humans, many disinfectants such as soap, bleach (sodium hypochlorite), surgical spirits, antiseptic, hand sanitizers, and hydrogen peroxide, are used to neutralize coronaviruses. Ultraviolet germicidal irradiation and steam sterilization with moist heat are used to decontaminate N95 face masks.

Authorized assays for viral testing include those that detect severe acute respiratory syndrome coronavirus 2 (SARS-CoV-2) nucleic acid or antigen. Viral (nucleic acid or antigen) tests check samples from the respiratory system (such as nasal swabs) and identify if an infection with SARS-CoV-2 is present. Viral tests are recommended to diagnose acute infection. Some tests are point-of-care tests, meaning results may be available at the testing location in less than an hour. Other tests must be sent to a laboratory to analyze, the results of which may take 1-2 days to evaluate once received by the laboratory. The diagnosis of coronavirus disease 2019 (COVID-19) requires detection of SARS-CoV-2 RNA by reverse transcription polymerase chain reaction (RT-PCR), which is more precise when nasopharynx samples are tested compared to throat samples.

A new wave of innovative diagnostic methods for virus detection have emerged and are listed in table 2.

TABLE 2

| | | ANALYTE/ | | |
|---|---|---|---|---|
| DEVICE | DEVELOPER | SAMPLE | DETECTION METHOD | PLATFORM |
| CARMEN- | Broad | Amplified | Color-coded droplets of | Microarray chip |
| Cas13a | Institute, | nucleic acid/Plasma, | samples are each paired | that contains |
| (Combinatorial | Harvard | nasal or | randomly in arrayed | 177,840 |
| Arrayed | University | throat swabs | microwells with color- | microwells and |
| Reactions for | | | coded droplets of | supports >4,500 |
| Multiplexed | | | CRISPR detection | statistically robust |
| Evaluation of | | | reagents, including | tests of |
| Nucleic Acids) | | | quenched fluorescent | crRNA: target pairs |
| | | | RNA reporters; detection | |
| | | | of target nucleic acid | |
| | | | results in Cas13a | |
| | | | activation, collateral | |
| | | | cleavage of the reporter | |
| | | | RNA species and the | |
| | | | generation of a | |
| | | | fluorescent signal, with a | |
| | | | time to result of <8 hours | |
| CRISPR-Chip | Cardea Bio | Unamplified | Label-free electrical | gFET connected to |
| | | nucleic | detection of a binding | portable digital |
| | | acid/Buccal | event between the target | reader |
| | | swab | sequence and an | |
| | | | immobilized, | |
| | | | catalytically deactivated | |
| | | | Cas9 enzyme complexed | |
| | | | with a target-specific | |
| | | | gRNA | |
| VIRRION | Pennsylvania | Whole virus/ | Carbon-nanotube-based | Chip containing |
| (virus capture | State | Nasopharyngeal | array for rapid size-based | nitrogen-doped |
| with rapid | University | swabs; exhaled | enrichment of viruses | carbon nanotube |
| Raman | | breath version | present in a sample | arrays decorated |
| spectroscopy | | in development | coupled with label-free, | with gold |
| detection and | | | non-destructive optical | nanoparticles to |
| identification) | | | detection using Raman | enhance |
| | | | spectroscopy | Raman |
| | | | | spectroscopy |
| | | | | signal |
| CRISPR- | University of | Unamplified | CRISPR-Cas13-based | Dry film |
| Cas13-based | Freiburg, | nucleic acid/ | detection of RNA that | photoresist layers |
| electrochemical | Germany | Serum | exploits non-specific | stacked on |
| microfluidic | | | collateral cleavage | a polyimide |
| sensor | | | activity of Cas13 for | substrate |
| | | | post-recognition signal | containing an |
| | | | amplification through a | electrochemical |
| | | | reporter RNA species; | cell for measuring |
| | | | uncleaved reporter RNA | hydrogen peroxide |
| | | | is recognized by | produced in |
| | | | antibodies bound to | inverse proportion |
| | | | glucose oxidase, which | to the amount of |
| | | | produces hydrogen | target analyte in |
| | | | peroxide, which in turn is | the sample |
| | | | detected by current | |
| | | | changes in an | |
| | | | electrochemical cell | |
| Convat optical | Catalan | Antigen in | Bimodal waveguide | All |
| biosensor | Institute of | point-of-care | interferometry; detects | instrumentation to |
| | Nano science | test format and | interference occurring | be integrated |
| | and | unamplified | between two modes of a | into a portable 25 × |
| | Nanotechnology | nucleic acid in | single light wave as it | 15 × 25 cm box |
| | (Barcelona, | multiplexed | interacts with an analyte | under tablet |
| | Spain) and | format/Nasal | bound to a sensing | control |
| | collaborators | or saliva swabs | element, such as an | |
| | | | antibody or a | |
| | | | complementary nucleic | |
| | | | acid strand | |
| Dual functional | Swiss Federal | Unamplified | Optical detection in 6-10 | Glass surface |
| plasmonic | Laboratories | label-free | minutes of viral RNA | supporting gold |
| photothermal | for Material | nucleic acid/ | hybridization with | nanoislands |
| biosensor | Science and | Bioaerosol | complementary DNA | functionalized with |
| | Technology, | | sequences immobilized | complementary |
| | Swiss Federal | | on gold nanoparticles, | DNA sequences |
| | Institute of | | employing localized | |
| | Technology in | | surface plasmon | |
| | Zurich (ETH | | resonance and plasmonic | |
| | Zurich) | | photothermal heating | |
| FET biosensor | Korea Basic | Antigen | Label-free, real-time | gFET linked to a |
| | Science | requiring no | electrical detection of | semiconductor |
| | Institute | sample | viral antigen binding | analyzer |
| | (Cheongju) | pretreatment/ | graphene-based FET | |
| | | Nasopharyngeal | functionalized with | |
| | | swabs | antibody; 100 | |
| | | | femtograms per milliliter | |
| | | | limit of detection | |
| FemtoSpot | Nano | Antiviral | Patient-operated | Change in |
| COVID-19 | DiagnosiX | immunoglobulin | serological test that uses | conductivity of a |
| Rapid | | G and M | electronic amplification | nanoribbon-based |
| Detection | | antibodies/One | to detect antibodies or | FET |
| Test | | drop of | disease biomarkers at | |
| | | untreated blood | low concentrations | |
| COVID-19 | University of | Viral antigen/ | Rapid one-minute test | Change in |
| biosensor | Utah | Saliva | using surface- | electrical |
| | | | immobilized | resistance |
| | | | oligonucleotide aptamers | |
| | | | to bind viral antigen | |
| One-step | Northwestern | Viral nucleic | Uses primer-free | Fluorescence read- |
| COVID-19 | University, | acid/Nasal or | CRISPR isothermal | out in less than |
| test | Stemloop | saliva swab; | amplification for one-pot | one hour |
| | | environmental | amplification and | |
| | | samples | detection of nucleic acid | |
| | | | at ambient temperature | |
| | | | with attomolar sensitivity | |

Unfortunately, the detection methods listed in Table 2 offer little help for people in public or private gatherings needing to find out immediately (i.e., in a few seconds) if the person is an asymptomatic virus carrier and may spread viruses to others. Moreover, capturing and disinfecting viruses is needed.

It would be advantageous to develop systems and methods for detecting, capturing and disinfecting viruses so that their spread may be curtailed.

### SUMMARY

Accordingly, a first embodiment of the present invention is directed to a water trap system comprising a water-filled container through which air, in the form of bubbles, is introduced, via an air pump, at a bottom of (or anywhere beneath the upper surface of the liquid) the water-filled container. Positively charged viruses (e.g., COVID-19), bacteria and other contaminants become trapped in the water-filled container as they are entrapped by negatively charged oxygen atoms. The water serves to filter (i.e., capture using water molecules) the virus from the air.

A second embodiment of the present invention is directed to the use of an electric field created by a pair of electrodes with an outer surface heated to a threshold temperature significant enough to disinfect viruses that come into contact therewith.

A third embodiment of the present invention is directed to a portable inhaler configured to detect the polarity of airborne virus and count the positive and negative electrical charges of airborne viruses with tolerances of a few charged particles per cubic centimeter by sampling several hundred cubic centimeters of air per second for a few seconds of time. Consequently, the portable inhaler is capable of quickly and efficiently detecting whether a person has contracted the COVID-19 virus.

Other variations, embodiments and features of the present invention will become evident from the following detailed description, drawings and claims.

### BRIEF DESCRIPTION OF THE DRAWINGS

FIG. 1 illustrates a perspective view of water container system according to the embodiments of the present invention;

FIG. 2 illustrates a cross-sectional view of the water container system of FIG. 1 according to the embodiments of the present invention;

FIG. 3 illustrates a perspective view of a system type for disinfecting virus according to the embodiments of the present invention;

FIG. 4 illustrates a cross-sectional view of the system type for disinfecting virus shown in FIG. 3 according to the embodiments of the present invention;

FIG. 5 illustrates a second cross-sectional view of the system type for disinfecting virus shown in FIG. 3 according to the embodiments of the present invention;

FIGS. 6A-6C illustrate an exemplary S-shaped air passageway configured to disinfect viruses according to the embodiments of the present invention;

FIG. 7 illustrates a portable inhaler for detecting virus according to the embodiments of the present invention;

FIG. 8 illustrates an interface for the portable inhaler according to the embodiments of the present invention;

FIG. 9 illustrates a cross-sectional view of the portable inhaler according to the embodiments of the present invention;

FIG. 10 illustrates an internal view of the components of the portable inhaler according to the embodiments of the present invention;

FIG. 11 illustrates a cross-sectional view of the portable inhaler with outer electrode designed in a cylindrical shape according to the embodiments of the present invention; and

FIG. 12 illustrates a cross-sectional view of the portable inhaler with outer electrode designed in an oval shape according to the embodiments of the present invention.

### DETAILED DESCRIPTION

For the purposes of promoting an understanding of the principles in accordance with the embodiments of the present invention, reference will now be made to the embodiments illustrated in the drawings and specific language will be used to describe the same. It will nevertheless be understood that no limitation of the scope of the invention is thereby intended. Any alterations and further modifications of the inventive feature illustrated herein, and any additional applications of the principles of the invention as illustrated herein, which would normally occur to one skilled in the relevant art and having possession of this disclosure, are to be considered within the scope of the invention claimed.

The individual parts of the various systems detailed herein may be made of any suitable materials including, but not limited to, metals, plastics, composites, alloys, polymers, and combinations thereof. The individual parts and components of the various systems detailed herein may be fabricated using suitable techniques including, but not limited to, molding, machining, rapid prototyping, casting and combinations thereof.

While the term “virus” is referenced below, those skilled in the art will recognize that the embodiments of the present invention may be used to detect, capture and/or kill bacteria and other airborne contaminants as well. The term “airborne pathogens” is used herein to describe the family of viruses, bacteria and containments. In broadest terms, the systems and methods detailed herein serve to detect, capture and/or kill airborne pathogens.

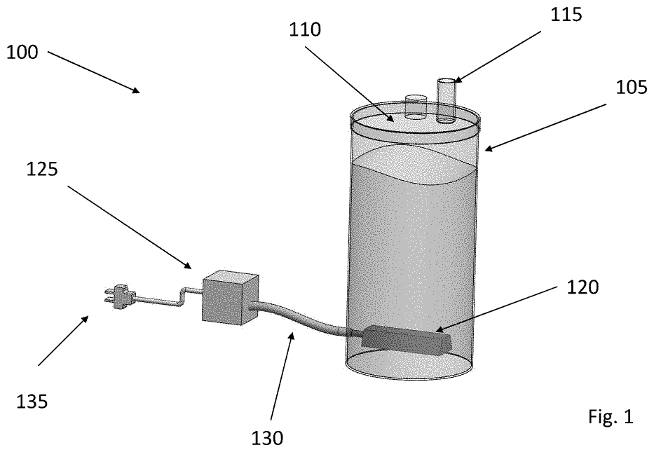

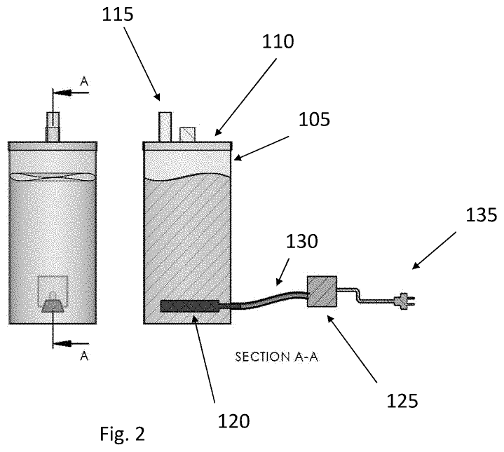

FIGS. 1 and 2 show perspective and cross-sectional views of a water container system 100 according to the embodiments of the present invention. The water container system 100 comprises a liquid (e.g., water) container 105, lid 110, air vent 115, air bubbler 120 and air pump 125. The air pump 125 communicates with the water container 105 via tubing 130. A power source 135 drives the air pump 125 to pump ambient air into the water container 105 via air bubbler 120. It is evident that the lid 110 may be a separate piece or may be integral with the liquid container 105.

In practice, the air pump 125 brings in air and forces it into the liquid container 105 via tubing 130. The air entering the water container 105 presumably contains airborne pathogens which need to be filtered out. Such filtering relies on water molecules which contain hydrogen and oxygen atoms held together by covalent bonds. Oxygen atoms carry a negative charge while hydrogen atoms carry a positive charge.

Coronaviruses, such as COVID-19 carry electrical charges (e.g., positive) on their surface spikes via arginine (C₆H₁₄N₄O₂)—an α-amino acid such that when the virus passes through the water in container 105, the individual viruses become entrapped by a plurality of oppositely charged oxygen atoms (e.g., negative). Consequently, clean filtered air passes though the water container 105 and released back into the environment via air vent 115 while the airborne pathogens are trapped in the water container 105.

In another embodiment, to enhance virus trapping efficiency in water, salts (NaCl) are added to form a solution rich in Na+ and Cl− ions, which, along with the oxygen atoms, trap oppositely charged viruses passing through the water. Other halogen elements, such as potassium iodide and fluorochlorobromide iodine, may also be added to enhance virus trapping efficiency in water. In general, the viruses may be passed through any suitable ionic compounds containing water or other liquids. In addition to water, other liquids and even solids with the correct polarity in their molecular structure may be utilized to entrap viruses.

In another embodiment, a detection device detects the polarity of viruses before they are forced into the liquid within container 105

The primary mode of transmission with COVID-19 is respiratory droplets that form when an infected person coughs or sneezes. Most cases of infection happen when people do not maintain a safe distance of 6 ft from each other. But, in cases where the infected person is in a small, enclosed place (airplane cabin or elevator), the virus can linger in the air for extended periods of time. In these cases, air needs to either be ventilated out or recirculated.

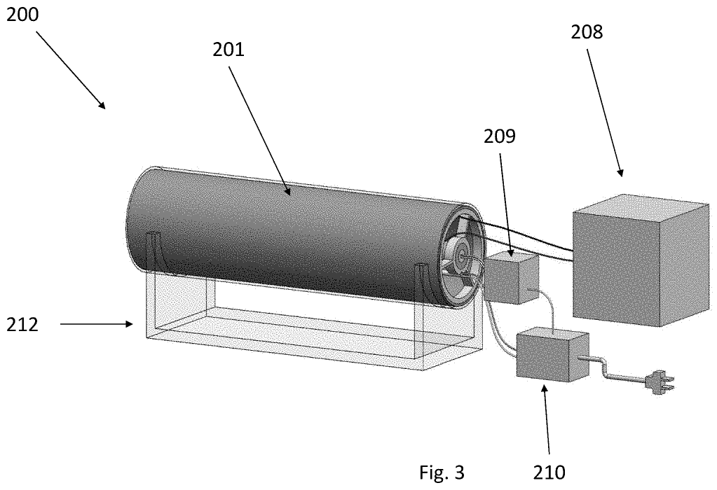

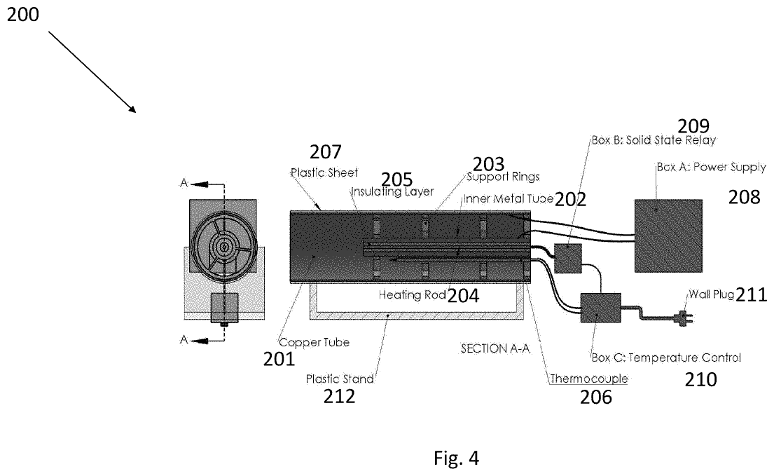

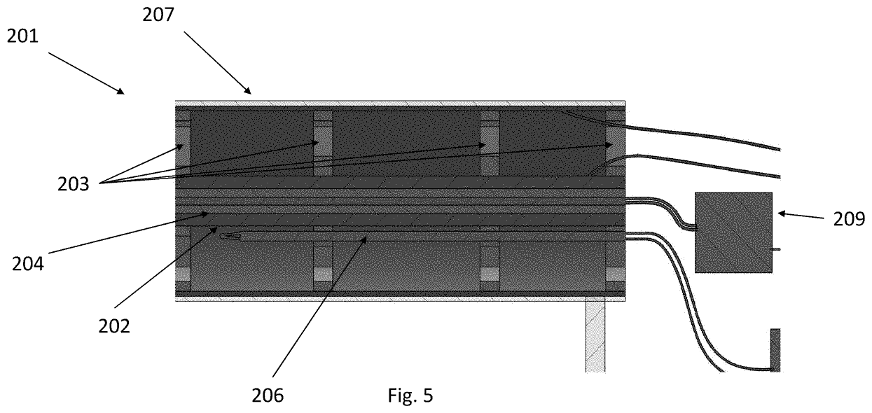

FIGS. 3-5 show a system 200 of the type for disinfecting viruses (i.e., air sanitizing device) according to the embodiments of the present invention. The system 200 functions in some respects akin to a Gerdien tube. A Gerdien tube is a device that measures the number or concentration of ions that are present in the air. This is accomplished using metal electrodes to attract the air ions. More specifically, a Gerdien tube consists of two electrodes (parallel to one another). There is an electric field between an inner electrode (the collector) and an outer electrode. The electric field is imposed by a voltage source. Negative air ions flowing through the Gerdien tube, impact the collector, and the current produced can be measured. The current measured is proportional to air ion concentration. The embodiments of the present invention use a similar concept to attract and kill viruses. A Gerdien tube 200 comprises an outer copper tube 201, inner metal tube 202, support rings 203, heating rod 204, insulating layer 205, thermocouple 206, external plastic covering 207, power supply 208, solid state relay 209, temperature control 210, wall plug 211 and stand 212.

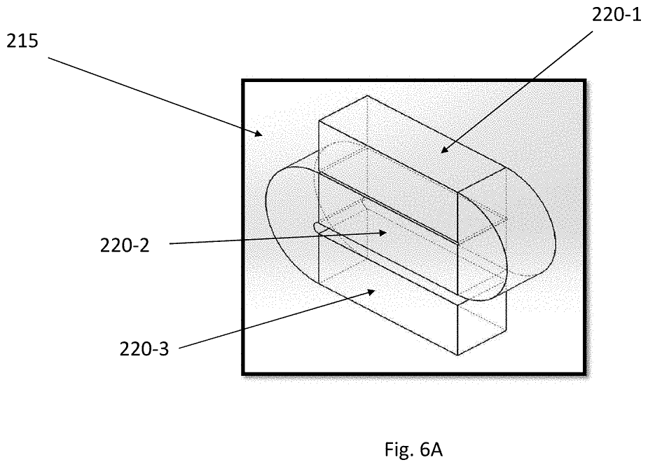

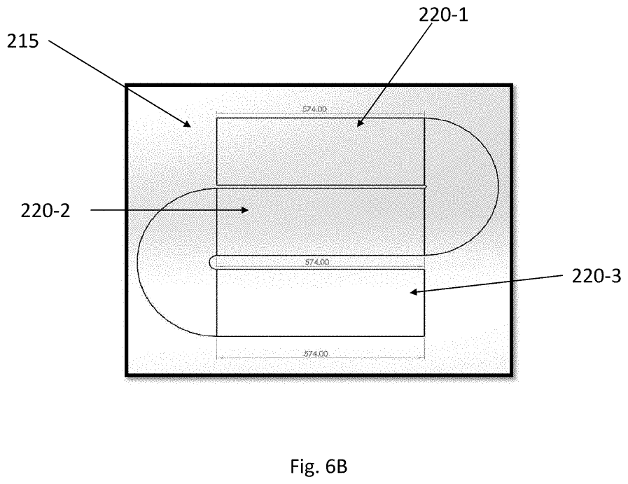

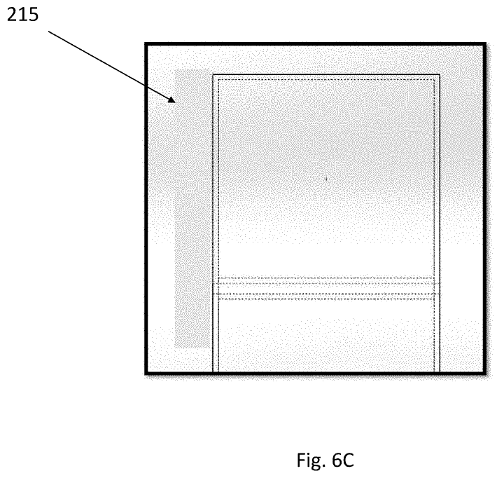

With the embodiments of the present invention, an air passageway (formed of ducting, tubing, piping, etc.) incorporates an inner electrode (collector) and outer electrode thereby creating an electric field within the air passageway. In this instance, the inner electrode is heated to a threshold temperature sufficient to disinfect a target virus upon contact. In one embodiment, the air passageway 215 is S-shaped as shown in FIGS. 6A-6C. Each straight section 220-1 through 220-3 of the air passageway 215 incorporates an outer and inner electrode with the inner electrode heated to the threshold temperature. As contaminated air is forced into the passageway 215, the viruses contact one of the inner electrodes along one of the straight sections and are killed. In one embodiment, the straight sections 220-1 through 220-3 measure 0.574 m resulting in a total length of 1.217 m. The outer electrode of the straight sections may be formed as part of the passageway or separate therefrom. For example, in one embodiment, the electrode may be a separate plate on an inner portion of the passageway along the straight sections 220-1 through 220-3.

For purposes of calculating system parameters, the specifications for SARS-CoV-2 virus were as follows: (i) a molecular weight of about 114 kDa (1.893*10⁻¹⁹ g); (ii) a molecule diameter ranging from 60-140 nm (1.13*10⁵-1.44*10⁶ nm³) and (iii) the accumulated positive charge on a Covid-19 virus caused by arginine (C₆H₁₄N₄O₂) on each spike is approximately 1.27×10⁻¹⁸, which may vary due to factors such as moisture and pH levels. Taking this into consideration, the inventor treats COVID-19 respiratory droplets as ions which attract to the collector electrode. For the safe fabrication of the system detailed herein, it is important to consider how long the electrodes need to be to properly remove all air contaminants; and the distance between the electrode plates be to maximize efficiency while also considering space limitations.

Since the mass (m) and charge (q) of Covid-19 is known, the inventor was able to measure the virus flowrate (v₀), from human breath for example, at the entry of the detecting device and capture the positively charged Covid-19 virus with a known electrical field (E) created by a pair of electrodes at a given voltage (V). The colliding distance (s) of a Covid-19 virus on the negatively charged electrode from the entry point is governed by the equations below.

E=V/d

F=q×E=m×a

F_{g}=m×g

s=v₀×d×(m/(Vq))^{1/2}

where F is the force on an airborne virus, a is acceleration on the airborne virus, g is gravity, v_{y} is velocity in the vertical (y) direction, d is distance between two electrodes, s is the 1^{st} colliding distance on the electrode.

In case the bottom electro-plate is negatively charged, a and g are in the same direction,

v_{y}=(2(a+g)*d/2)^{1/2}

s₁=v₀*(2*d/(2/(a+g))^{1/2}

s₂=s₁+(2*e*v_{y}/(a+g))*v₀

s₃=s₂+(2*e*e*v_{y}/(a+g))*v₀

s₄=s₃+(2*e*e*e*v_{y}/(a+g))*v₀

Where s₁ is the 1^{st} colliding distance on the electrode, s₂ is the 2^{nd} colliding distance after bouncing, s₃ is the 3^{rd} colliding distance after 2^{nd} bounce if it happens, s₄ is the 4^{th} colliding distance after 3^{rd} bounce if it happened, and e is coefficient of restitution, e=v_{y′}/v_{y} (The coefficient of restitution is the ratio of the final to the initial relative velocity between two objects after they collide. In this instance, the virus particles bounce off a stationary electrode)

In case the top electro-plate is negatively charged, a and g are in the opposite directions,

v_{y′}=(2(a−g)*d/2)^{1/2}

s₁=v₀*(2*d/(2/(a−g))^{1/2}

s₂=s₁+(2*e*v_{y}/(a−g))*v₀

s₃=s₂+(2*e*e*v_{y}/(a−g))*v₀

s₄=s₃+(2*e*e*e*v_{y}/(a−g))*v₀

In case different airborne viruses enter the device, their different mass and charge lead to different colliding distances on the electrode from Covid-19 viruses, hence they can be detected/separated by their colliding distances. If two (or more) viruses possess the same type of charge (both positive or both negative), they collide on the same electrode at different distances and hence are separated and can be identified. If two viruses possess different charges, they collide on different electrodes at distances governed by the equations given above. Using colliding distances on each electrode, airborne viruses can be fingerprinted accordingly.

In a real test environment, many airborne viruses from human breath contain some level of moisture, which alters their mass (weight) and only a portion of them collide at the distance given by equation 1. The rest are trapped in aerosols, which are mostly in size of microns or larger. A filter/mask with pores, such as of 0.3 micron, can be placed at the entry point of the device to block aerosols but allow airborne viruses to pass. In another embodiment, a pair of different pore-sized filters allow certain sized aerosols to pass through the filters to enter the detecting device. Therefore, equation 1 can be used to identify viruses.

The air damping effect can be considered in the calculation of colliding distance for Covid-19 with the formula below, to identify the actual colliding distance for viruses with different moistures.

F_{D}=(½)·C_{D}·ρv²

Where:

TABLE 3

F_{D}: damping force in Newton on the object
C_{D}: Coefficient of damping (no unit)
A: Area of the object facing the fluid in m²
ρ: Density of the fluid in kg/m³
v: Terminal velocity of the object
m: mass of the object in kg
g: Acceleration due to gravity in m/s²
a: Acceleration due to electrostatic force in m/s²
q: Charge in C
E: Electric field intensity in V/m

Table 4 is a calculation of the maximum distance, using the equations above, until the viruses inevitably collide with the electrode. In Table 4, viruses such as SARS, HPV-5, HPV-16, Influenza A, and Influenza B were calculated in addition to Covid-19 virus. Properties for each virus are given in Table 5.

TABLE 4

| | SARS Cov-2 | SARS Cov-1 | HPV-5 | HPV-16 | Influenza A | Influenza B |
|---|---|---|---|---|---|---|
| Electrode plate | Negatively | Negatively | Positively | Negatively | Negatively | Negatively |
| of collision | charged | charged | charged | charged | charged | charged |
| Velocity of virus | 0.5 | 0.5 | 0.5 | 0.5 | 0.5 | 0.5 |
| particles (v0) (m/s) | | | | | | |
| Voltage between | 5 | 5 | 5 | 5 | 5 | 5 |
| the plates (V) | | | | | | |
| Distance between | 20 | 20 | 20 | 20 | 20 | 20 |
| electrode plates | | | | | | |
| (d) (mm) | | | | | | |
| Distances at which | 5.11 | 4.33 | 2.68 | 2.57 | 7.74 | 6.01 |
| virus particles | | | | | | |
| collide with | | | | | | |
| electrode plate (mm) | | | | | | |

TABLE 5

| Feature | SARS Cov-2 | SARS Cov-1 | HPV-5 | HPV-16 | Influenza A | Influenza B |
|---|---|---|---|---|---|---|
| Diameter (D) (nm) | 60 | 80 | 52 | 52 | 80 | 100 |
| Volume (Vol) (nm3) | 113100 | 268000 | 73600 | 73600 | 6700 | 520000 |
| Mass (m) (kg) | 1.6605E−18 | 2.989E−19 | 9.86E−20 | 9.86E−20 | 8.00E−19 | 2.90E−19 |
| Accumulated | 1.2708E−18 | 3.177E−19 | 2.74E−19 | 2.98E−19 | 2.67E−19 | 1.60E−19 |
| charge on | | | at pH = 7.4 | at pH = 7.4 | | |
| the virus (C) | | | | | | |

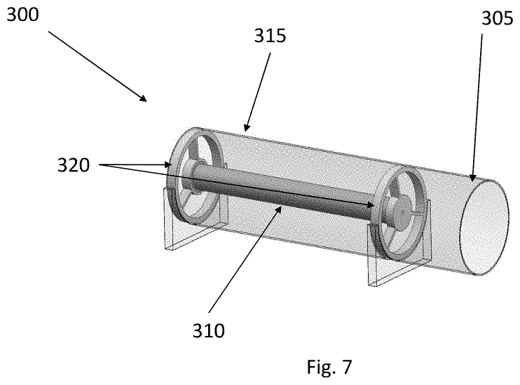

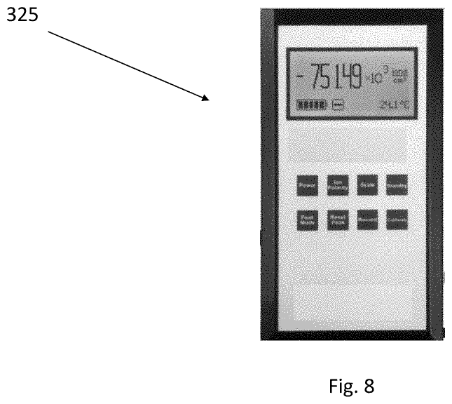

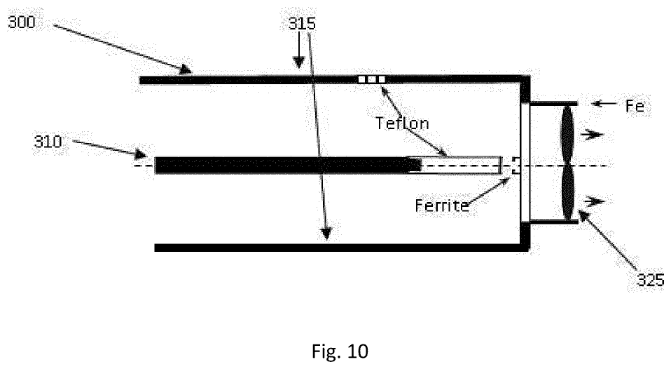

FIGS. 7-12 show a portable inhaler 300 configured to locate asymptomatic virus carrier(s) in public or private gatherings within a very short time (e.g., a few seconds). The inhaler 300 may also be used to analyze indoor or outdoor air for the presence of airborne viruses. The inhaler 300 comprises a tubular housing 305, an inner electrode 310 (collector), an outer electrode 315 support 320 rings and a voltage source. In one embodiment, inner electrode 310 and outer electrode 315 are parallel to one another. The outer electrode 315 may be formed as part of the tubular housing 305 or separate therefrom. For example, in one embodiment, the electrode may be a separate plate on an inner portion of the tubular housing 305. The inhaler 300 may also incorporate a user interface 325 which provides data and allows the user to input data, calibrate the device and so on.

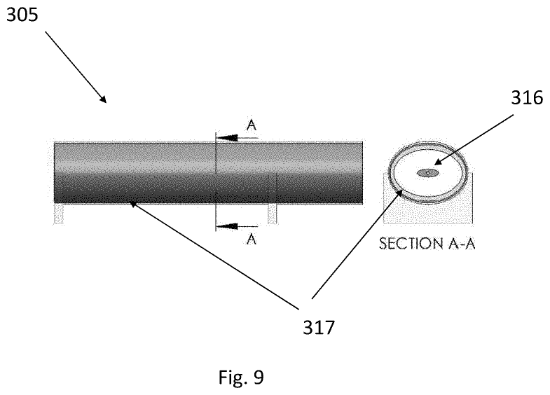

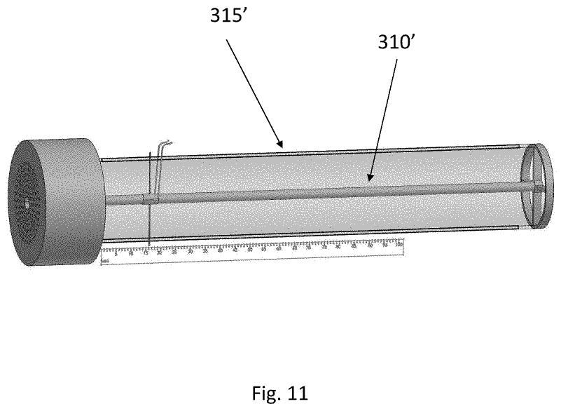

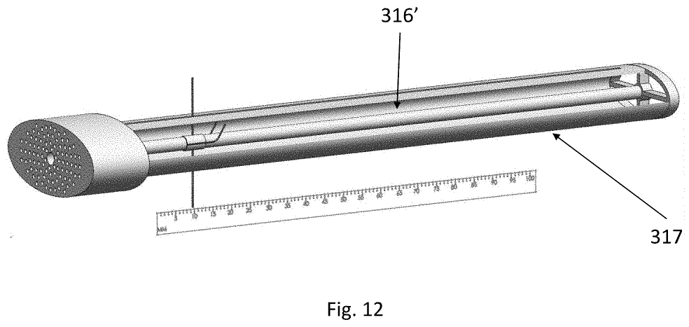

In one embodiment, as shown in FIGS. 7 and 11, the inner electrode 310, 310′ and outer electrode 315, 315′ have circular cross sections but, as shown in FIGS. 9 and 12, inner electrode 316. 316′ and outer electrode 317, 317′ may take on an elliptical shape, which leads to laminar air flow with reduced turbulence, therefore, providing improved measurement accuracy. The surfaces of the electrodes 310, 310′, 315, 315′, 316, 316′, 317 and 317′ are to be formed as smooth as possible by processes such as grinding and lapping to achieve accurate measurement. An applied voltage from the voltage source 320 creates an electric field between the inner electrode 310, 310′, 316 and 316′ and the outer electrode 315, 315′, 317 and 317′, respectively. Within the tubular housing 305, viruses of the same charge as the polarizing voltage are repelled by the outer electrode 315 as they move from a fan 325 through the tubular housing 305, and eventually move into the electric field contacting the inner electrode 310 causing a small current measured by a pA-meter. The measured current is proportional to virus concentration. This process is the same for each of the devices shown in FIGS. 7, 9, 11 and 12.

Since the currents detected are extremely low (e.g., 10⁻¹⁰ to 10⁻¹⁵ A), it is important to eliminate or significantly reduce the influence of ambient electric charge. This is accomplished using an active shielding to obtain high insulating resistance wherein the active shielding is generated by an electromagnetic field produced by the circuitry of the system. The active shielding increases the insulating resistance of the polarizing voltage source and leakage resistance of the inner and outer electrodes.

For purposes of experimentation: (i) a known amount of human breath (M) is blown by fan 325 through the tubular housing 305 of the inhaler 300 and (ii) the inner electrode 310 was polarized by a DC adjustable voltage (U) so an electrical field with nonhomogeneous intensity appears. In this scenario, positively charged viruses are attracted to the negatively charged inner electrode 310. As one virus impacts the inner electrode 310 a current (I) is generated. Because of the high value of inner impedance of the inner electrode 310, the value of I is small and measured by an electrometer. When the voltage (U) is high enough, the current (I) is saturated and directly proportional to the virus concentration, which can be obtained by solving equation:

$$n = \frac{I}{M \cdot e}$$

where n is the virus concentration in breath (charge·m⁻³); M=S·v is volume rate flow of breath through the aspiration condenser (m³s⁻¹); S=π(r₂²−r₁²) is area of cross-section of the condenser (m²); r₂, r₁ are diameters of outer and inner electrodes (m); e is charge of an electron or a positron, 1.602·10⁻¹⁹.

Those skilled in the art will recognize that leakage resistances RAK of the inhaler tubular housing 305, leakage resistances and capacitance of the pA-meter input (REH, CEH, REL, CEL), and insulation resistance (RV) of the inhaler collector voltage source 320 are important factors in the system design in order to reduce errors in current measurement. In addition, the current measured is also affected by the input resistance of pA-meter and the input resistance of voltage source (RU, CU) 320. In general, RAK and RV should be much larger than RI, and REH, and REL should be much larger than ROUT to minimize the measurement error. Also, time constant RUCU needs to be much larger than the measuring time.

In one embodiment, as the measured current intensity depends on polarization voltage, which is related to the dimension and parameters of inhaler tubular housing 305 and virus concentration level, and often in the range of 10⁻¹⁰ A-10⁻¹⁵ A, a transimpedance amplifier is used for the conversion and amplification.

In one embodiment, the transimpedance amplifier can be realized with an INA 116 op amp. The INA 116 has low input bias current Ib,max=100 fA. The first stage has transimpedance RT=10 GΩ. The second stage is a variable-gain amplifier. The gain is set by resistor RG. The resulting current-to-voltage conversion constant can be set to 0.1-1-10 pA/V.

Although the invention has been described in detail with reference to several embodiments, additional variations and modifications exist within the scope and spirit of the invention as described and defined in the following claims.

I claim:

1. A system for removing airborne pathogens from ambient air comprising:
a container for holding a liquid;
an air vent near a top of said container;
a bubbler positioned below a surface of said liquid;
an air pump configured to force air into said liquid in said container via said bubbler; and
wherein atoms of a liquid within said container entrap atoms of airborne pathogens entering said container with said air thereby removing them from the air.

2. The system of claim 1 wherein said liquid is water.

3. The system of claim 1 wherein said liquid incudes halogen elements.

4. The system of claim 1 wherein said liquid is water and said airborne pathogens are coronaviruses.

5. The system of claim 1 wherein said air vent is configured to allow filtered air to exit said container.

6. A system for detecting and disinfecting airborne pathogens comprising:
an air passageway;
an inner electrode within said air passageway;
an outer electrode within said air passageway;
a voltage source connected to one or both of said inner electrode and outer electrode to create an electric field therebetween;
means for heating said inner electrode;
means for forcing air into said air passageway; and
wherein said inner electrode is heated to a temperature to disinfect subject airborne pathogens coming into contact therewith.

7. The system of claim 6 further comprising a thermocouple.

8. The system of claim 6 further comprising a temperature control for controlling a temperature of said inner electrode.

9. The system of claim 6 wherein said inner electrode and outer electrode have a circular cross section.

10. The system of claim 6 wherein said inner electrode and outer electrode have an elliptical cross section.

11. The system of claim 6 wherein said air passageway is S-shaped.

12. The system of claim 6 further comprising active shielding generated by an electromagnetic field produced by circuitry of the system.

13. The system of claim 6 further comprising transimpedance amplifier.

14. An inhaler device for detecting airborne pathogens in a person's breath comprising:
a tubular housing;
an inner electrode within said tubular housing;
an outer electrode within said tubular housing;
a voltage source connected to one or both of said inner electrode and outer electrode to create an electric field therebetween; and
means to measure a current generated on said inner electrode by viruses impacting the same.

15. The system of claim 14 further comprising means to draw air into said tubular housing.

16. The system of claim 14 wherein said inner electrode and outer electrode have a circular cross section.

17. The system of claim 14 wherein said inner electrode and outer electrode have an elliptical cross section.
